(19) United States
(12) Patent Application Publication
(10) Pub. No.: US 2026/0277100 A1
(43) Pub. Date: Sep. 17, 2026

(54) SEMICONDUCTOR PHOTORESIST COMPOSITION AND METHOD OF FORMING PATTERNS USING THE COMPOSITION

(71) Applicant: SAMSUNG SDI CO., LTD., Yongin-si (KR)

(72) Inventors: Miyeon HAN, Yongin-si (KR); Bukeun OH, Yongin-si (KR); Jaeyeol BAEK, Yongin-si (KR); Jung Min CHOI, Yongin-si (KR); Seol Hee LIM, Yongin-si (KR); Seunghwan BYUN, Yongin-si (KR); Hyejin JANG, Yongin-si (KR); Jonghyuk JEON, Yongin-si (KR); Sanghoon SHIN, Yongin-si (KR)

(21) Appl. No.: 19/441,468

(22) Filed: Jan. 6, 2026

(30) Foreign Application Priority Data
Mar. 17, 2025 (KR) 10-2025-0034123

(51) Int. Cl. G03F 7/004; C07C 43/13; C07F 7/22; C07H 3/02; G03F 7/00; G03F 7/16; G03F 7/20; H10P 50/00; H10P 76/20

(52) U.S. Cl. CPC G03F 7/0045; C07C 43/132; C07C 43/135; C07F 7/2224; C07H 3/02; G03F 7/0042; G03F 7/168; G03F 7/2004; G03F 7/70033; H10P 76/20; H10P 50/71; H10P 50/73

(57) ABSTRACT

A semiconductor photoresist composition and a method of forming patterns using the semiconductor photoresist composition are provided. The semiconductor photoresist composition includes an organometallic compound, a compound including four or more hydroxyl groups and one or more ether bonds, and a solvent.

### CROSS-REFERENCE TO RELATED APPLICATION

The present application claims priority to and the benefit of Korean Patent Application No. 10-2025-0034123, filed on Mar. 17, 2025, in the Korean Intellectual Property Office, the entire disclosure of which is incorporated herein by reference.

### BACKGROUND

### 1. Field

One or more embodiments of the present disclosure relate to a semiconductor photoresist composition and a method of forming patterns using the same.

### 2. Description of the Related Art

Extreme ultraviolet (EUV) lithography has emerged as an important technology for manufacturing next-generation semiconductor devices, such as advanced semiconductor chips (e.g., next generation semiconductor chips). EUV lithography utilizes EUV radiation with a wavelength of 13.5 nanometers (nm) as an exposure light source, enabling the formation of extremely fine patterns, for example, patterns having critical dimensions of 20 nm or less.

Achieving high-resolution patterning with EUV lithography requires the development of compatible photoresists capable of sub-16 nm resolution. However, chemically amplified (CA) photoresists currently face limitations in resolution, photospeed, and line edge roughness (LER), which hinder their performance in advanced lithographic processes.

In CA photoresists, acid-catalyzed reactions may cause image blurring, particularly at small feature sizes, a limitation also observed in electron beam lithography. Although CA photoresists are designed for high sensitivity, their typical elemental composition results in low absorbance at 13.5 nm, thereby reducing sensitivity under EUV exposure.

Additionally, CA photoresists often exhibit increased LER as photospeed decreases, due in part to the stochastic nature of acid diffusion and reaction. These limitations create the need for novel, high-performance photoresist materials suitable for EUV lithography.

In response, research has turned to inorganic photoresist compositions, which are primarily used for negative tone patterning. These compositions undergo chemical modification through non-chemically amplified mechanisms, providing resistance to developer solutions. Inorganic photoresists typically contain elements with higher EUV absorption than hydrocarbons, offering improved sensitivity, reduced stochastic effects, and lower LER.

Inorganic photoresists based on peroxopolyacids of tungsten, optionally mixed with elements, such as niobium, titanium, and/or tantalum, have been explored as radiation sensitive materials for patterning.

These materials have demonstrated efficacy in patterning large-pitch features using bilayer configurations and various radiation sources, including deep UV, X-ray, and electron beam. For example, cationic hafnium metal oxide sulfate (HfSOx) materials, when combined with a peroxo complexing agent, have enabled imaging of 15 nm half-pitch features via EUV projection exposure. While this system offers high performance and acceptable photospeed, it suffers from practical drawbacks: (i) the coating process involves corrosive sulfuric acid/hydrogen peroxide mixtures, leading to poor shelf-life stability; (ii) structural modifications for performance enhancement are difficult; and (iii) development requires highly concentrated tetramethylammonium hydroxide (TMAH) solutions (e.g., 25 wt %).

To address these issues and challenges, recent efforts have focused on tin-containing molecules with strong EUV absorption. Among these, organotin polymers have shown promise. Upon EUV exposure, alkyl ligands dissociate and form oxo bonds with adjacent chains, enabling negative tone patterning resistant to organic developers. Although these organotin polymers exhibit improved sensitivity, resolution, and LER, further enhancements are needed or desired to meet commercial performance requirements.

### SUMMARY

One or more aspects of embodiments of the present disclosure are directed toward a semiconductor photoresist composition capable of improving sensitivity, storage stability, and coatability.

One or more aspects of embodiments of the present disclosure are directed toward a method of forming patterns using the semiconductor photoresist composition.

Additional aspects will be set forth in part in the description which follows and, in part, will be apparent from the description, or may be learned by practice of the presented embodiments of the disclosure.

According to one or more embodiments of the present disclosure, a semiconductor photoresist composition includes: an organometallic compound; a compound including four or more hydroxyl groups and one or more ether bonds; and a solvent.

According to one or more embodiments of the present disclosure, a method of forming patterns includes: forming an etching-objective layer (e.g., etching-target layer) on a substrate; applying the semiconductor photoresist composition to the etching-objective layer to form a photoresist film; patterning the photoresist film to form a photoresist pattern; and etching the etching-objective layer using the photoresist pattern as an etching mask.

The semiconductor photoresist composition according to one or more embodiments of the present disclosure has excellent or suitable sensitivity, storage stability, and coatability, and when the semiconductor photoresist composition is used, a small-sized pattern may be precisely controlled or formed, and excellent or suitable resolution may be implemented.

For example, one or more embodiments of the present disclosure provide a semiconductor photoresist composition that combines (i) an organometallic compound—such as an organotin species having at least one oxygen-containing ligand for enhanced EUV absorption and crosslinking capability—with (ii) a polyhydroxyl ether compound that improves coating uniformity and storage stability, and (iii) a suitable solvent system. This synergistic formulation addresses limitations of comparable chemically amplified resists by delivering high sensitivity, reduced line width roughness, and robust pattern fidelity at sub-20 nm critical dimensions under EUV exposure. Methods and systems for forming patterns may utilize the composition, to enable precise, high-aspect-ratio patterning for advanced semiconductor device fabrication.

### BRIEF DESCRIPTION OF DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIGS. 1A-1E are cross-sectional views for explaining a method of forming patterns using a semiconductor photoresist composition according to one or more embodiments of the present disclosure.

### DETAILED DESCRIPTION

The present disclosure may be modified in many alternate forms, and thus specific embodiments will be exemplified in the drawing and described in more detail. It should be understood, however, that it is not intended to limit the present disclosure to the particular forms disclosed, but rather, is intended to cover all modifications, equivalents, and alternatives falling within the teachings and scope of the present disclosure.

Hereinafter, referring to the drawings, one or more embodiments of the present disclosure will be described in more detail. In the following description of the present disclosure, the well-established functions or constructions will not be described in order to make the present disclosure concise.

To clearly illustrate the present disclosure, certain unessential description and relationships are omitted, and throughout the disclosure, the same or similar configuration elements are designated by the same reference numerals. Also, because the size and thickness of each configuration shown in the drawing are illustratively shown for better understanding and ease of description, the present disclosure is not necessarily limited thereto.

In the drawings, the thickness of layers, films, panels, regions, and/or the like, may be enlarged or reduced for clarity. In the drawings, the thickness of a part of layers or regions, and/or the like, may be exaggerated or reduced for convenience of explanation. It will be understood that if (e.g., when) an element such as a layer, film, region, or substrate is referred to as being “on” another element, it may be directly on the other element or one or more intervening elements may also be present therebetween. In contrast, when an element is referred to as being “directly on” another element, there are no intervening elements present therebetween.

The terminology used herein is used to describe embodiments only, and is not intended to limit the present disclosure. The singular expression includes the plural expression unless the context clearly dictates otherwise. For example, the singular forms “a,” “an,” “one,” and “the” as used herein are intended to include the plural forms as well unless the context clearly indicates differently. Further, the utilization of “may” when describing embodiments of the present disclosure refers to “one or more embodiments of the present disclosure”.

As used herein, “combination thereof” refers to a mixture, a laminate, a composite, a copolymer, an alloy, a blend, a reaction product, and/or the like of the constituents.

Here, the term “and/or” or “or” is not to be construed as an exclusive meaning, for example, “A and/or B” or “A or B” is construed to include A, B, A+B, and/or the like.

Here, it should be understood that terms such as “comprise(s)/comprising,” “include(s)/including,” and/or “has(have)/having” are intended to designate the presence of an embodied feature, number, step, element, or a combination thereof, but it does not preclude the possibility of the presence or addition of one or more other features, numbers, steps, elements, or a combination thereof. Additionally, the terms “comprise(s)/comprising,” “include(s)/including,” “have/has/having,” or other similar terms include or support the terms “consisting of” and “consisting essentially of,” indicating the presence of stated features, integers, steps, operations, elements, and/or components, without or essentially without the presence of other features, integers, steps, operations, elements, components, and/or groups thereof. Further, the use of “may” when describing embodiments of the present disclosure refers to “one or more embodiments of the present disclosure.

As used herein, “substituted” refers to replacement of a hydrogen by deuterium, a halogen, a hydroxyl group, a carboxyl group, a thiol group, a cyano group, a nitro group, —NRR′ (wherein, R and R′ may each independently be hydrogen, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group), —SiRR′R″ (wherein, R, R′, and R″ may each independently be hydrogen, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group), a C1 to C30 alkyl group, a C1 to C10 haloalkyl group, a C1 to C10 alkylsilyl group, a C3 to C30 cycloalkyl group, a C6 to C30 aryl group, a C1 to C20 alkoxy group, a C1 to C20 sulfide group, or a combination thereof. “Unsubstituted” refers to non-replacement of a hydrogen by another substituent and remaining of the hydrogen.

As used herein, if (e.g., when) a definition is not otherwise provided, the term “alkyl group” refers to a linear or branched aliphatic hydrocarbon group. The alkyl group may be a “saturated alkyl group” without any double bond or triple bond.

The alkyl group may be a C1 to C8 alkyl group. For example, the alkyl group may be a C1 to C7 alkyl group, a C1 to C6 alkyl group, or a C1 to C5 alkyl group. For example, the C1 to C5 alkyl group may be a methyl group, an ethyl group, a propyl group, an isopropyl group, an n-butyl group, an isobutyl group, a sec-butyl group, a tert-butyl group, or a 2,2-dimethylpropyl group.

As used herein, if (e.g., when) a definition is not otherwise provided, the term “cycloalkyl group” refers to a monovalent cyclic aliphatic hydrocarbon group.

The cycloalkyl group may be a C3 to C8 cycloalkyl group, for example, a C3 to C7 cycloalkyl group, or a C3 to C6 cycloalkyl group. For example, the cycloalkyl group may be a cyclopropyl group, a cyclobutyl group, a cyclopentyl group, or a cyclohexyl group, but embodiments of the present disclosure are not limited thereto.

As used herein, the term “aryl group” refers to a substituent in which all atoms in the cyclic substituent have a p-orbital and these p-orbitals are conjugated and may include a monocyclic functional group or a fused ring polycyclic functional group (i.e., rings sharing adjacent pairs of carbon atoms).

As used herein, the term “heteroaryl group” may refer to an aryl group including at least one heteroatom selected from among N, O, S, P, and Si. Two or more heteroaryl groups may be linked by a sigma bond directly, or if (e.g., when) the heteroaryl group includes two or more rings, the two or more rings may be fused. When the heteroaryl group is a fused ring, one or more rings thereof may include one to three heteroatoms.

As used herein, the term “arylalkyl group” refers to a substituent in which one or more hydrogens of an alkyl group are replaced by an aryl group.

As used herein, the term “alkenyl group” refers to a linear or branched aliphatic hydrocarbon group including at least one double bond as an aliphatic unsaturated alkenyl group.

As used herein, the term “alkynyl group” refers to a linear or branched aliphatic hydrocarbon group including at least one triple bond as an aliphatic unsaturated alkynyl group.

Hereinafter, a semiconductor photoresist composition according to one or more embodiments will be described.

A semiconductor photoresist composition according to one or more embodiments may include an organometallic compound, a compound including four or more hydroxyl groups and one or more ether bonds, and a solvent.

A general organic photoresist composition in the art has no high (e.g., low) etch resistance, and thus if an aspect ratio is increased, there is a problem and issue of deteriorating resolution of a pattern. In addition, an inorganic photoresist composition in the art, which is vulnerable to moisture and has inferior storage stability, has a problem and issue of causing particle-shaped defects during a coating process.

The semiconductor photoresist composition according to one or more embodiments includes a compound including four or more hydroxyl groups and one or more ether bonds, so that stability with a coating solution or a coating film may be improved, achieving excellent or suitable coatability. In addition, the semiconductor photoresist composition, of which sensitivity is also improved, may realize excellent or suitable pattern formability.

The compound including four or more hydroxyl groups and one or more ether bonds may include (e.g., be) one or more compounds selected from among a saturated or unsaturated aliphatic compound and an aromatic compound, each including four or more hydroxyl groups and one or more ether bonds.

In one or more embodiments, the compound including four or more hydroxyl groups and one or more ether bonds may be a compound represented by Chemical Formula 1 or Chemical Formula 2:

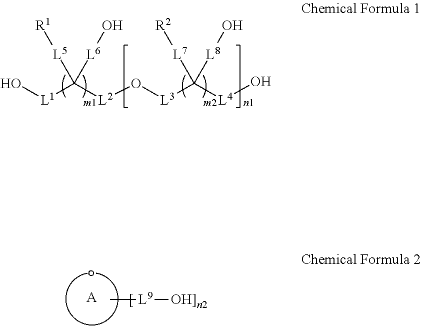

In Chemical Formula 1 and Chemical Formula 2,
L¹ to L⁹ may each independently be a single bond or a substituted or unsubstituted C1 to C10 alkylene group,
R¹ and R² may each independently be any one selected from among hydrogen, a hydroxyl group, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C2 to C10 heteroalkenyl group, a substituted or unsubstituted C2 to C10 heteroalkynyl group, a substituted or unsubstituted C3 to C20 cycloalkane, a substituted or unsubstituted C3 to C20 cycloalkene, a substituted or unsubstituted C6 to C20 aromatic ring, and a substituted or unsubstituted C3 to C20 hetero aromatic ring,
A (e.g., ring A) may be any one selected from among a substituted or unsubstituted C4 to C20 oxacycloalkane and a substituted or unsubstituted C4 to C20 oxacycloalkene,
m1 and m2 may each independently be one of the integers from 1 to 10,
n1 may be one of the integers from 1 to 10, and
n2 may be an integer greater than or equal to 4 and less than or equal to a valence of A (e.g., ring A).

In one or more embodiments, in Chemical Formula 1, R¹ and R² may each independently be any one selected from among hydrogen, a hydroxyl group, a substituted or unsubstituted C1 to C10 alkyl group, and a substituted or unsubstituted C3 to C20 cycloalkane, for example, may be any one selected from among hydrogen, a hydroxyl group, a substituted or unsubstituted C1 to C8 alkyl group, and a substituted or unsubstituted C3 to C15 cycloalkane, or, for example, may be any one selected from among hydrogen, a hydroxyl group, a substituted or unsubstituted C1 to C5 alkyl group, and a substituted or unsubstituted C3 to C10 cycloalkane.

In one or more embodiments, in Chemical Formula 2, A may be a substituted or unsubstituted C4 to C10 oxacycloalkane, for example, a substituted or unsubstituted C4 to C9 oxacycloalkane, or a substituted or unsubstituted C4 to C7 oxacycloalkane.

In one or more embodiments, in Chemical Formula 1 and Chemical Formula 2, L¹ to L⁹ may each independently be a single bond or a substituted or unsubstituted C1 to C8 alkylene group, for example, a single bond or a substituted or unsubstituted C1 to C7 alkylene group, or a single bond or a substituted or unsubstituted C1 to C5 alkylene group.

In one or more embodiments, in Chemical Formula 1, m1 and m2 may each independently be one of the integers from 1 to 8, for example, one of the integers from 1 to 5, or one of the integers from 1 to 3.

In one or more embodiments, in Chemical Formula 1, n1 may be an integer from 1 to 8, for example, an integer from 1 to 5, or an integer from 1 to 3.

In one or more embodiments, in Chemical Formula 2, n2 may be an integer from 4 to 15, for example, an integer from 4 to 10, or an integer from 4 to 8.

In one or more embodiments, in Chemical Formula 1 and Chemical Formula 2, L¹ to L⁹ may each independently be a single bond or a substituted or unsubstituted C1 to C5 alkylene group, R¹ and R² may each independently be any one selected from among hydrogen, a hydroxyl group, a substituted or unsubstituted C1 to C5 alkyl group, and a substituted or unsubstituted C3 to C10 cycloalkane, A may be a substituted or unsubstituted C4 to C7 oxacycloalkane, m1 and m2 may each independently be one of integers of 1 to 3, n1 may be an integer from 1 to 3, and n2 may be an integer from 4 to 8.

In one or more embodiments, the compound including four or more hydroxyl groups and one or more ether bonds may be a compound represented by any one selected from among Chemical Formula 1-1 to Chemical Formula 1-5:

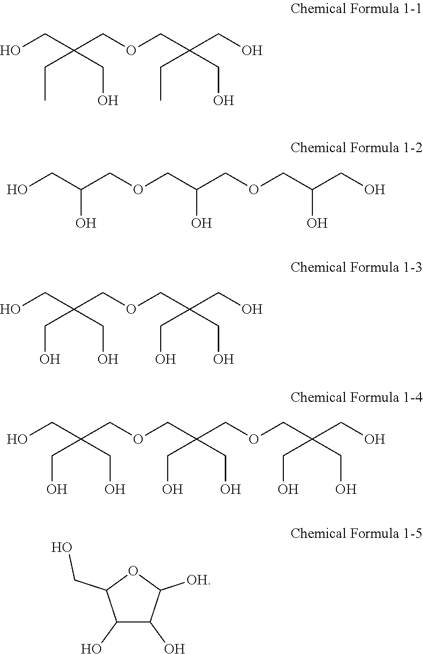 [SMILES: CCC(CO)(CO)COCC(CC)(CO)CO ; OCC(O)COCC(O)COCC(O)CO ; OCC(CO)(CO)COCC(CO)(CO)CO ; OCC(CO)(CO)COCC(CO)(CO)COCC(CO)(CO)CO ; OCC1OC(O)C(O)C1O]

The compound including four or more hydroxyl groups and one or more ether bonds may be included in an amount of about 0.001 wt % to about 10 wt %, for example, about 0.005 wt % to about 8 wt %, about 0.01 wt % to about 5 wt %, about 0.01 wt % to about 3 wt %, about 0.01 wt % to about 1 wt %, or about 0.05 wt % to about 1 wt % based on a total weight of 100 wt % of the semiconductor photoresist composition. When the amount of the compound including four or more hydroxyl groups and one or more ether bonds satisfies the above range, sensitivity, storage stability, and coatability of the semiconductor photoresist composition may be further improved.

The organometallic compound may be included in an amount of about 0.5 wt % to about 30 wt %, for example, about 1 wt % to about 30 wt %, about 1 wt % to about 25 wt %, for example, about 1 wt % to about 20 wt %, for example, about 1 wt % to about 15 wt %, for example, about 1 wt % to about 10 wt %, for example, about 1 wt % to about 5 wt % based on the total weight of 100 wt % of the semiconductor photoresist composition. When the amount of the organometallic compound satisfies the above range, the sensitivity of the photoresist may be improved. In addition, if (e.g., when) the amount of the organometallic compound satisfies the above range, the pattern formability may be improved while the influence of nitrogen oxides is reduced.

In one or more embodiments, the organometallic compound may be an organotin compound containing at least one of an organic oxy group and an organic carbonyloxy group.

The organometallic compound may be represented by Chemical Formula 3:

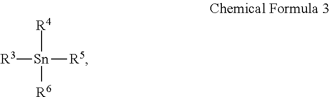

In Chemical Formula 3,
R³ may be selected from among a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and a substituted or unsubstituted C7 to C30 arylalkyl group,
R⁴ to R⁶ may each independently be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, a substituted or unsubstituted C7 to C30 arylalkyl group, an alkoxy or aryloxy group (—OR^{a}, wherein R^{a} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl or acyloxy group (—O(CO)R^{b}, wherein R^{b} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylamido or dialkylamido group (—NR^{c}R^{d}, wherein R^{c} and R^{d} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidato group (—NR^{e}(COR^{f}), wherein R^{e} and R^{f} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidinato group (—NR^{g}C(NR^{h})Rⁱ, wherein R^{g}, R^{h}, and Rⁱ may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylthio or arylthio group (—SR^{j}, wherein R^{j} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or a thiocarboxyl group (—S(CO)R^{k}, wherein R^{k} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and
at least one selected from among R⁴ to R⁶ may be selected from among an alkoxy group and an aryloxy group (—OR^{a}, wherein R^{a} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and a carboxyl group and an acyloxy group (—O(CO)R^{b}, wherein R^{b} may be selected from among hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

In one or more embodiments, R⁴ to R⁶ may each independently selected from among an alkoxy group and an aryloxy group (—OR^{a}, wherein R^{a} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and a carboxyl group and an acyloxy group (—O(CO)R^{b}, wherein R^{b} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

In one or more embodiments, because the organometallic compound represented by Chemical Formula 3 includes —OR^{a} and/or —O(CO)R^{b} as ligand(s), a pattern formed using a semiconductor photoresist composition including the organometallic compound may exhibit excellent or suitable limit resolution.

Additionally, the ligand(s) of —OR^{a} and/or —O(CO)R^{b} may determine the solubility of the organometallic compound represented by Chemical Formula 3 in a solvent.

In one or more embodiments, R³ may be a substituted or unsubstituted C1 to C8 alkyl group, a substituted or unsubstituted C3 to C8 cycloalkyl group, a substituted or unsubstituted C2 to C8 aliphatic unsaturated organic group including one or more double bonds or triple bonds, a substituted or unsubstituted C6 to C20 aryl group, a substituted or unsubstituted C4 to C20 heteroaryl group, a carbonyl group, an ethoxy group, a propoxy group, or a combination thereof,
R^{a} may be a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof, and
R^{b} may be hydrogen, a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof.

In one or more embodiments, R³ may be a methyl group, an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, a formyl group, an acetyl group, a propanoyl group, a butanoyl group, a pentanoyl group, an ethoxy group, a propoxy group, or a combination thereof,
R^{a} may be an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, or a combination thereof, and
R^{b} may be hydrogen, an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, or a combination thereof.

In one or more embodiments, the organometallic compound may be represented by Chemical Formula 4 or Chemical Formula 5:

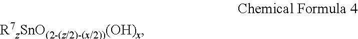

wherein, in Chemical Formula 4,
R⁷ may be a C1 to C31 hydrocarbyl group, 0<z≤2, and 0<(z+x)≤4,

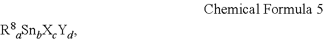

wherein, in Chemical Formula 5,
R⁸ may be a substituted or unsubstituted C1 to C20 alkyl group, a substituted or unsubstituted C3 to C20 cycloalkyl group, a substituted or unsubstituted C2 to C20 aliphatic unsaturated organic group including one or more double bonds or triple bonds, a substituted or unsubstituted C6 to C30 aryl group, a substituted or unsubstituted C4 to C30 heteroaryl group, a carbonyl group, an ethylene oxide group, a propylene oxide group, or a combination thereof,
X may be sulfur (S), selenium (Se), or tellurium (Te),
Y may be —OR^{m} or —OC(═O)Rⁿ,
wherein R^{m} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof, and
Rⁿ may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof, and
a, b, c, and d may each independently be an integer of 1 to 20.

The solvent included in the semiconductor photoresist composition according to one or more embodiments may be an organic solvent. For example, the solvent may be, for example, selected from among aromatic compounds (e.g., xylene, toluene, and/or the like), alcohols (e.g., 4-methyl-2-pentanol, 4-methyl-2-propanol, 1-butanol, methanol, isopropyl alcohol, 1-propanol), ethers (e.g., anisole, tetrahydrofuran), esters (n-butyl acetate, propylene glycol monomethyl ether acetate, ethyl acetate, ethyl lactate), ketones (e.g., methyl ethyl ketone, 2-heptanone), and/or a mixture thereof, but embodiments of the present disclosure are not limited thereto.

The semiconductor resist composition according to one or more embodiments may further include a resin in addition to the aforementioned organometallic compound, compound including four or more hydroxyl groups and one or more ether bonds, and solvent.

The resin may be a phenol-based resin including at least one aromatic moiety listed (e.g., at least one selected from the moieties) in Group 1.

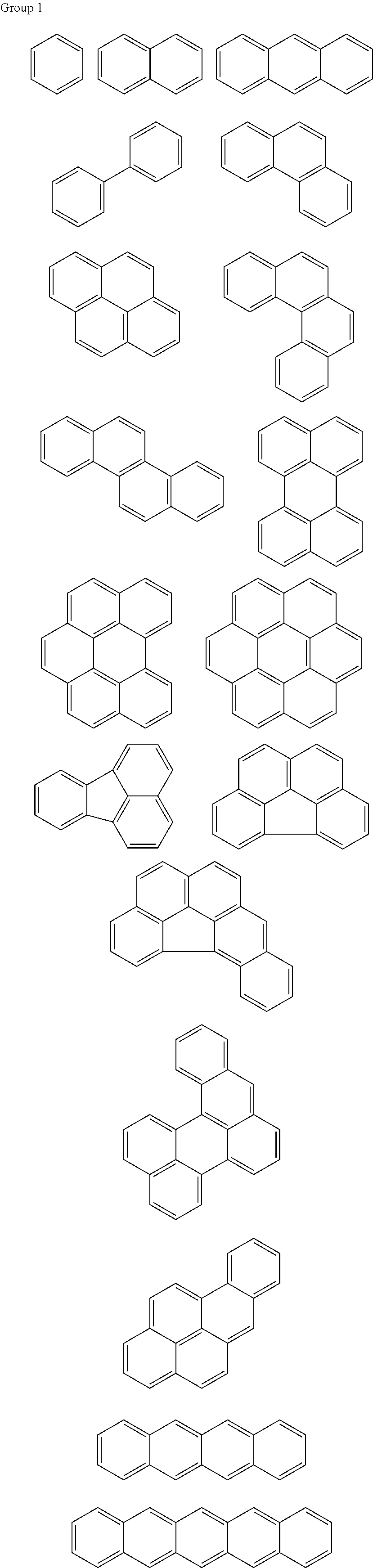

The resin may have a weight average molecular weight of about 500 g/mol to about 20,000 g/mol.

The resin may be included in an amount of about 0.1 wt % to about 50 wt % based on a total amount of 100 wt % of the semiconductor photoresist composition. If (e.g., when) the resin is included in the above amount range, the semiconductor photoresist composition may have excellent or suitable etch resistance and heat resistance.

According to one or more embodiments of the present disclosure, the semiconductor photoresist composition may be composed of the aforementioned organometallic compound, compound including four or more hydroxyl groups and one or more ether bonds, solvent, and resin.

According to one or more embodiments of the present disclosure, the semiconductor photoresist composition may further include one or more additives selected from among a surfactant, a crosslinking agent, a leveling agent, organic acid, a quencher, and a combination thereof, as needed.

The surfactant may include, for example, an alkyl benzene sulfonate salt, an alkyl pyridinium salt, polyethylene glycol, a quaternary ammonium salt, and/or a combination thereof, but embodiments of the present disclosure are not limited thereto.

The crosslinking agent may be, for example, a melamine-based crosslinking agent, a substituted urea-based crosslinking agent, an acrylic crosslinking agent, an epoxy-based crosslinking agent, a polymer-based crosslinking agent, or a combination thereof, but embodiments of the present disclosure are not limited thereto. In one or more embodiments, it may be a crosslinking agent having at least two crosslinking forming substituents, for example, a compound such as methoxymethylated glycoluril, butoxymethylated glycoluril, methoxymethylated melamine, butoxymethylated melamine, methoxymethylated benzoguanamine, butoxymethylated benzoguanamine, 4-hydroxybutyl acrylate, acrylic acid, urethane acrylate, acryl methacrylate, 1,4-butanediol diglycidyl ether, glycidol, diglycidyl 1,2-cyclohexane dicarboxylate, trimethylpropane triglycidyl ether, 1,3-bis(glycidoxypropyl)tetramethyldisiloxane, methoxymethylated urea, butoxymethylated urea, methoxymethylated thiourea, and/or the like.

The leveling agent may be used for improving coating flatness during printing and may be a commercially available suitable leveling agent.

The organic acid may include p-toluenesulfonic acid, benzenesulfonic acid, p-dodecylbenzenesulfonic acid, 1,4-naphthalenedisulfonic acid, methanesulfonic acid, a fluorinated sulfonium salt, malonic acid, citric acid, propionic acid, methacrylic acid, oxalic acid, lactic acid, glycolic acid, succinic acid, and/or a combination thereof, but embodiments of the present disclosure are not limited thereto.

The quencher may include diphenyl (p-tolyl) amine, methyl diphenyl amine, triphenyl amine, phenylenediamine, naphthylamine, diaminonaphthalene, and/or a combination thereof, but embodiments of the present disclosure are not limited thereto.

An amount of each of the additives included in the semiconductor photoresist composition may be controlled or selected depending on desired or suitable properties.

In one or more embodiments, the semiconductor photoresist composition may further include a silane coupling agent as an adherence enhancer in order to improve a close-contacting force with a substrate (e.g., in order to improve adherence of the semiconductor photoresist composition to the substrate). The silane coupling agent may include, for example, a silane compound including a carbon-carbon unsaturated bond such as vinyltrimethoxysilane, vinyl triethoxysilane, vinyl trichlorosilane, vinyl tris(β-methoxyethoxy)silane; 3-methacryloxypropyltrimethoxysilane, 3-acryloxypropyltrimethoxysilane, p-styryl trimethoxysilane, 3-methacryloxypropylmethyldimethoxysilane, 3-methacryloxypropylmethyl diethoxysilane; trimethoxy[3-(phenylamino)propyl]silane; and/or the like, but embodiments of the present disclosure are not limited thereto.

The semiconductor photoresist composition may be formed into a pattern having a high aspect ratio without a collapse. Accordingly, in order to form a fine pattern having a width (e.g., line width) of, for example, about 5 nm to about 100 nm (e.g., to form the fine pattern having a critical dimension (CD) of about 5 nm to about 100 nm), for example, about 5 nm to about 80 nm, for example, about 5 nm to about 70 nm, for example, about 5 nm to about 50 nm, for example, about 5 nm to about 40 nm, for example, about 5 nm to about 30 nm, or for example, about 5 nm to about 20 nm, the semiconductor photoresist composition may be used for a photoresist process using light in a wavelength in a range of about 5 nm to about 150 nm, for example, about 5 nm to about 100 nm, about 5 nm to about 80 nm, about 5 nm to about 50 nm, about 5 nm to about 30 nm, or about 5 nm to about 20 nm. Accordingly, the semiconductor photoresist composition according to one or more embodiments may be used to realize extreme ultraviolet lithography using an EUV light source of a wavelength of about 13.5 nm.

According to one or more embodiments, a method of forming patterns using the aforementioned semiconductor photoresist composition is provided. For example, the manufactured pattern may be a photoresist pattern.

The method of forming patterns according to one or more embodiments includes forming an etching-objective layer (e.g., etching-target layer) on a substrate; applying the semiconductor photoresist composition to the etching-objective layer to form a photoresist film; patterning the photoresist film to form a photoresist pattern; and etching the etching-objective layer using the photoresist pattern as an etching mask.

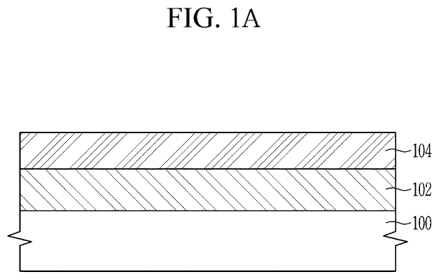

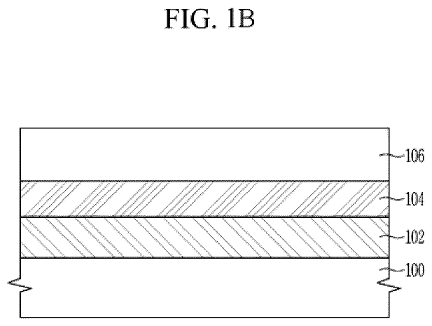

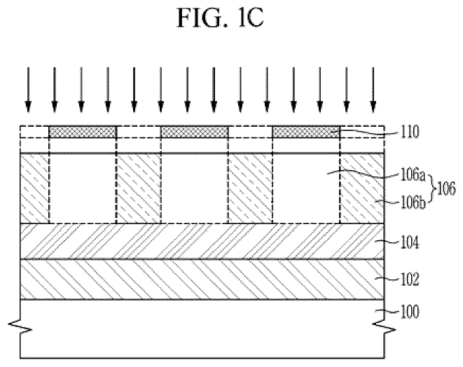

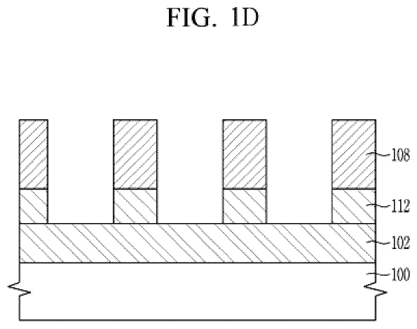

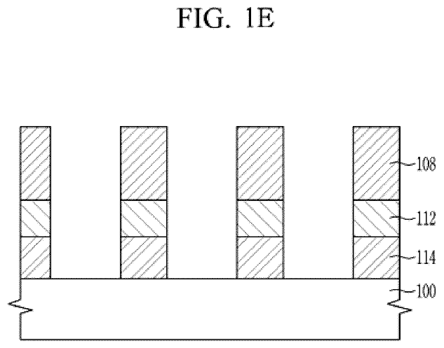

Hereinafter, a method of forming patterns using the aforementioned semiconductor photoresist composition will be described in more detail referring to FIGS. 1A-1E. FIGS. 1A-1E are cross-sectional views for illustrating a method of forming patterns using a semiconductor photoresist composition according to one or more embodiments of the present disclosure.

Referring to FIG. 1A, an object for etching (e.g., etching-objective layer or etching-target layer) is prepared. The object for etching may be a thin film 102 formed on a semiconductor substrate 100. Hereinafter, the object for etching is limited to the thin film 102. A surface of the thin film 102 is washed to remove impurities and/or the like remaining thereon. The thin film 102 may be, for example, a silicon nitride layer, a polysilicon layer, or a silicon oxide layer.

Subsequently, a resist underlayer composition for forming a resist underlayer 104 is spin-coated on the surface of the washed thin film 102. However, embodiments of the present disclosure are not limited thereto, and various suitable coating methods, for example, a spray coating, a dip coating, a knife edge coating, a printing method (for example, an inkjet printing and/or a screen printing), and/or the like may be used.

In one or more embodiments, the coating process of the resist underlayer may not be provided, but hereinafter, a process including a coating of the resist underlayer is described.

Then, the coated resist underlayer composition is dried and baked to form the resist underlayer 104 on the thin film 102. The baking may be performed at about 100° C. to about 500° C., for example, about 100° C. to about 300° C.

The resist underlayer 104 is formed between the substrate 100 and a photoresist film 106 and thus may prevent or reduce non-uniformity of pattern formability of a photoresist line width if (e.g., when) a ray reflected from on the interface between the substrate 100 and the photoresist film 106 or a hardmask between layers is scattered into an unintended photoresist region.

Referring to FIG. 1B, the photoresist film 106 is formed by coating the semiconductor photoresist composition on the resist underlayer 104. The photoresist film 106 is obtained by coating the aforementioned semiconductor photoresist composition on the thin film 102 formed on the substrate 100 and then, curing it through a heat treatment.

In one or more embodiments, the formation of a pattern by using the semiconductor photoresist composition may include coating the semiconductor resist composition on the substrate 100 having the thin film 102 through spin coating, slit coating, inkjet printing, and/or the like and then, drying it to form the photoresist film 106.

The semiconductor photoresist composition has already been illustrated in more detail and will not be illustrated again.

Subsequently, the substrate 100 having the photoresist film 106 is subjected to a first baking process. The first baking process may be performed at about 80° C. to about 120° C.

Referring to FIG. 1C, the photoresist film 106 may be selectively exposed using a patterned mask 110.

For example, the exposure may use an activation radiation with light having a high energy wavelength light such as EUV (extreme ultraviolet; a wavelength of about 13.5 nm), an E-Beam (an electron beam), and/or the like as well as light such as an i-line (a wavelength of about 365 nm), a KrF excimer laser (a wavelength of about 248 nm), an ArF excimer laser (a wavelength of about 193 nm), and/or the like.

In one or more embodiments, light or exposure beam for the exposure may be light having a wavelength in a range of about 5 nm to about 150 nm and/or a high energy wavelength, for example, EUV (extreme ultraviolet; a wavelength of 13.5 nm), and/or may be an E-Beam (an electron beam), and/or the like.

The exposed region 106b of the photoresist film 106 has a different solubility from the unexposed region 106a of the photoresist film 106 by forming a polymer by a crosslinking reaction such as condensation between organometallic compounds.

Subsequently, the substrate 100 is subjected to a second baking process. The second baking process may be performed at a temperature of about 90° C. to about 200° C. The exposed region 106b of the photoresist film 106 becomes indissoluble regarding a developer due to the second baking process.

In FIG. 1D, the unexposed region 106a of the photoresist film is dissolved and removed using the developer to form a photoresist pattern 108. For example, the unexposed region 106a of the photoresist film is dissolved and removed by using an organic solvent such as 2-heptanone and/or the like to complete the photoresist pattern 108 corresponding to a negative tone image.

As described above, the developer used in the method of forming patterns according to one or more embodiments may be an organic solvent. The organic solvent used in the method of forming patterns according to one or more embodiments may include, for example, a ketone such as methylethylketone, acetone, cyclohexanone, 2-heptanone, and/or the like, an alcohol such as 4-methyl-2-propanol, 1-butanol, isopropanol, 1-propanol, methanol, and/or the like, an ester such as propylene glycol monomethyl ether acetate, ethyl acetate, ethyl lactate, n-butyl acetate, butyrolactone, and/or the like, an aromatic compound such as benzene, xylene, toluene, and/or the like, or a combination thereof.

However, the photoresist pattern according to one or more embodiments is not necessarily limited to the negative tone image but may be formed to have a positive tone image. In this case, suitable positive-tone developers include quaternary ammonium hydroxide compositions (solutions) such as tetramethylammonium hydroxide (TMAH), tetraethylammonium hydroxide, tetrapropylammonium hydroxide, and/or tetrabutylammonium hydroxide, individually or in combination.

As described above, exposure to light having a high energy such as EUV (extreme ultraViolet; a wavelength of 13.5 nm), to an E-Beam (an electron beam), and/or the like, and/or to light such as i-line (wavelength of about 365 nm), KrF excimer laser (wavelength of about 248 nm), ArF excimer laser (wavelength of about 193 nm), and/or the like may provide a photoresist pattern 108 having a width of a thickness of about 5 nm to about 100 nm. For example, in one or more embodiments, the photoresist pattern 108 may have a width of a thickness of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm.

In one or more embodiments, the photoresist pattern 108 may have a pitch (center-to-center distance between adjacent features in the pattern) having (or with) a half-pitch of less than or equal to about 50 nm, for example less than or equal to about 40 nm, for example less than or equal to about 30 nm, for example less than or equal to about 20 nm, or for example less than or equal to about 15 nm, and a line width roughness of less than or equal to about 10 nm, less than or equal to about 5 nm, less than or equal to about 3 nm, or less than or equal to about 2 nm.

Subsequently, the photoresist pattern 108 is used as an etching mask to etch the resist underlayer 104. Through this etching process, an organic film pattern 112 is formed. The organic film pattern 112 may also have a width corresponding to that of the photoresist pattern 108.

Referring to FIG. 1E, the exposed thin film 102 is etched by applying the photoresist pattern 108 as an etching mask. As a result, the thin film is formed as a thin film pattern 114.

The etching of the thin film 102 may be performed by dry etching using, for example, an etching gas, and the etching gas may be, for example, CHF₃, CF₄, Cl₂, BCl₃, or a mixed gas thereof.

In the exposure process, the thin film pattern 114 formed by using the photoresist pattern 108 formed through the exposure process performed by using an EUV light source may have a width corresponding to that of the photoresist pattern 108. For example, the thin film pattern 114 may have a width (e.g., line width) of about 5 nm to about 100 nm which is equal to that of the photoresist pattern 108. For example, in one or more embodiments, the thin film pattern 114 formed by using the photoresist pattern 108 formed through the exposure process performed by using an EUV light source may have a width (e.g., line width) of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm, for example, a width (e.g., line width) of less than or equal to about 20 nm, like that of the photoresist pattern 108.

Hereinafter, the present disclosure will be described in more detail through examples of the preparation of the aforementioned semiconductor photoresist composition. However, the present disclosure is technically not restricted by the following examples.

### Synthesis of Organometallic Compounds

### Synthesis Example 1

40.7 g of t-butylSnPh₃ and 300 g of propionic acid were added to a 250 mL two-necked round-bottom flask and heated under reflux for 24 hours. Unreacted propionic acid was removed under reduced pressure to obtain a compound represented by Chemical Formula 6.

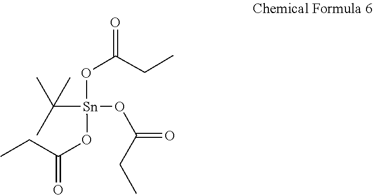 [SMILES: CCC(=O)[O][Sn]([O]C(=O)CC)([O]C(=O)CC)[C](C)(C)C]

### Synthesis Example 2

30 mL of anhydrous pentane was added to 10 g of t-AmylSnCl₃, the temperature was maintained at 0° C., and then 7.4 g of diethylamine and 6.1 g of ethanol were added thereto, and stirred at room temperature for 1 hour. When the reaction was completed, the resultant was filtered, concentrated, and vacuum-dried to obtain a compound represented by Chemical Formula 7.

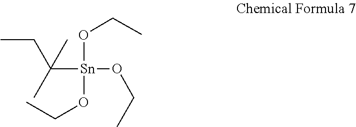

### Synthesis Example 3

10 g of dibutyltin dichloride was dissolved 30 mL of ether, 70 mL of a 1 M sodium hydroxide (NaOH) aqueous solution was added thereto and then, stirred for 1 hour. After the stirring, a solid produced therein was filtered, three times washed with 25 mL of deionized water, and dried at 100° C. under a reduced pressure to obtain an organometallic compound represented by Chemical Formula 8 and having a weight average molecular weight of 1,500 g/mol.

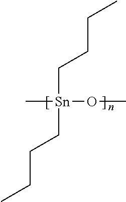

### Preparation of Semiconductor Photoresist Compositions

### Examples and Comparative Examples

The organometallic compounds represented by Chemical Formulas 6 to 8 according to Synthesis Examples 1 to 3 were respectively dissolved at a concentration of 3 wt % in propylene glycol methyl ether acetate (PGMEA). Subsequently, the compounds represented by Chemical Formulas 1-1 to 1-5 and 9 to 11 were respectively added and dissolved therein in each amount shown in Table 1 (based on a total weight of 100 wt % of a semiconductor photoresist composition) and then, filtered with a 0.1 μm PTFE (polytetrafluoroethylene) syringe filter, preparing respective semiconductor photoresist compositions according to Examples and Comparative Examples.

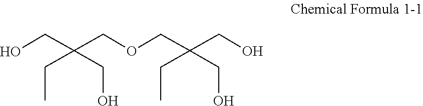 [SMILES: CCC(CO)(CO)COCC(CC)(CO)CO]

(Di(trimethylolpropane), Manufacturer: Sigma-Aldrich Co., Ltd.)

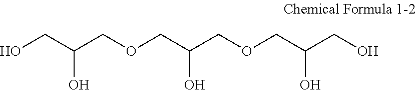 [SMILES: OCC(O)COCC(O)COCC(O)CO]

(Triglycerol, manufacturer: Sigma-Aldrich Co., Ltd.)

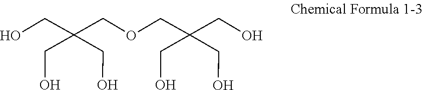 [SMILES: OCC(CO)(CO)COCC(CO)(CO)CO]

(Dipentaerythritol, Manufacturer: Sigma-Aldrich Co., Ltd.))

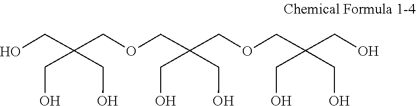 [SMILES: OCC(CO)(CO)COCC(CO)(CO)COCC(CO)(CO)CO]

(Tripentaerythritol, Manufacturer: Sigma-Aldrich Co., Ltd.)

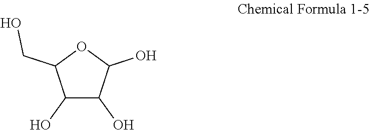

(D-(−)-ribose, Manufacturer: Sigma-Aldrich Co., Ltd.)

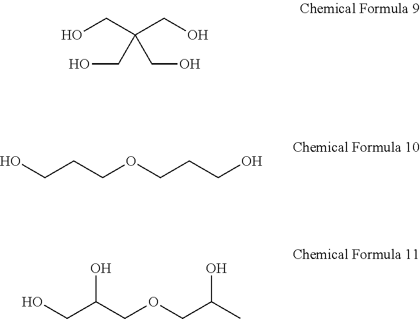

TABLE 1

| | | | Amount of | |
|---|---|---|---|---|
| | Type of | | organometallic | Amount of |
| | organometallic | Type of | compound | compound |
| | compound | compound | (wt %) | (wt %) |
| Example 1 | Chemical | Chemical | 2.9 | 0.1 |
| | Formula 7 | Formula 1-1 | | |
| Example 2 | Chemical | Chemical | 2.9 | 0.1 |
| | Formula 7 | Formula 1-2 | | |
| Example 3 | Chemical | Chemical | 2.9 | 0.1 |
| | Formula 7 | Formula 1-3 | | |
| Example 4 | Chemical | Chemical | 2.9 | 0.1 |
| | Formula 7 | Formula 1-4 | | |
| Example 5 | Chemical | Chemical | 2.9 | 0.1 |
| | Formula 7 | Formula 1-5 | | |
| Example 6 | Chemical | Chemical | 2.9 | 0.1 |
| | Formula 6 | Formula 1-1 | | |
| Example 7 | Chemical | Chemical | 2.9 | 0.1 |
| | Formula 8 | Formula 1-1 | | |
| Comparative | Chemical | — | 3.0 | — |
| Example 1 | Formula 7 | | | |
| Comparative | Chemical | Chemical | 2.9 | 0.1 |
| Example 2 | Formula 7 | Formula 9 | | |
| Comparative | Chemical | Chemical | 2.9 | 0.1 |
| Example 3 | Formula 7 | Formula 10 | | |
| Comparative | Chemical | Chemical | 2.9 | 0.1 |
| Example 4 | Formula 7 | Formula 11 | | |

### Evaluation 1: Sensitivity Evaluation

As used herein, sensitivity denotes the EUV exposure dose (in mJ/cm²) required to resolve a 50-nm critical dimension (CD) negative-tone line/space pattern at the specified process conditions. Each of the semiconductor photoresist compositions was coated to be 240 angstroms (Å) thick on a silicon wafer and then, processed through PAB (post-apply bake), exposure, PEB (post-exposure bake), and development processes, forming patterned films.

Each of the semiconductor photoresist compositions according to the examples and the comparative examples was spin-coated at 1,500 rpm for 30 seconds on a 200 mm circular silicon wafer of which the surface was deposited with hexamethyldisilazane (HMDS), baked (post-apply baked (PAB)) at 110° C. for 60 seconds, and allowed to stand at room temperature (23±2° C.) for 30 seconds, thereby preparing a respective coated wafter.

Subsequently, a 50 nm-wide straight line array was projected onto the wafer coated with the semiconductor photoresist composition by using EUV light (MET, Lawrence Berkeley National Laboratory Micro Exposure Tool). Herein, pad exposure time was adjusted, so that an EUV dose was increasing applied to each pad of the array.

The resist film and the substrate (i.e., silicon wafer) were exposed at 170° C. for 120 seconds on a hotplate and then, baked (post-exposure bake (PEB)). The baked film was immersed in a developing solution (2-heptanone) for 30 seconds and washed with the same developer additionally for 10 seconds to from a negative tone image, that is, to remove a nonexposed portion of the coating. Finally, the process was completed by baking at 150° C. for 2 minutes on a hot plate.

The resist film was measured with respect to a line width to exposed dose (energy) changes by using Critical Dimension Scanning Electron Microscope (CD-SEM). The resist line widths differently formed according to each exposure dose were used to measure appropriate or suitable sensitivity, and the results are shown in Table 2.

### Evaluation 2: Storage Stability Evaluation

The semiconductor photoresist compositions according to the examples and the comparative examples were stored in a vial under room temperature and normal pressure conditions to examine whether precipitates were formed or not for 5 days with naked eyes, which were used to evaluate storage stability according to the following criteria, and the results are shown in Table 2.

### Evaluation Criteria for Storage Stability

x: If there is precipitate
∘: If no precipitate is observed

### Evaluation 3: Coating Characteristics Evaluation

Each of the semiconductor photoresist compositions according to the examples and the comparative examples was spin-coated at 1500 rpm for 60 seconds on a wafer and baked at 110° C. for 60 seconds to form a respective thin film, of which images were taken with an atomic force microscope (AFM) and/or the like to measure surface roughness of the thin film by using a software (ex. optical profiler). Among the surface roughnesses, a root mean square roughness (R^{q}) refers to a root mean square (rms) of a vertical value within a reference length of the roughness profile. Based on the measured surface roughness, coating characteristics were evaluated according to the following criteria, and the results are shown in Table 2.

### Evaluation Criteria for Coating Characteristics

⊚: R^{q} value is less than or equal to 0.3 nm
∘: R^{q} value is greater than 0.3 nm and less than or equal to 0.4 nm
x: R^{q} value is greater than 0.4 nm

TABLE 2

| | Sensitivity | Storage | Coating |
|---|---|---|---|
| | (mJ/cm²) | stability | characteristics |
| Example 1 | 31.2 | ◯ | ⊚ |
| Example 2 | 30.9 | ◯ | ⊚ |
| Example 3 | 31.5 | ◯ | ⊚ |
| Example 4 | 32.5 | ◯ | ⊚ |
| Example 5 | 34.1 | ◯ | ◯ |
| Example 6 | 35.0 | ◯ | ◯ |
| Example 7 | 33.6 | ◯ | ◯ |
| Comparative Example 1 | 53 | X | X |
| Comparative Example 2 | 50 | ◯ | X |
| Comparative Example 3 | 51.2 | ◯ | X |
| Comparative Example 4 | 52.3 | ◯ | X |

It was confirmed that the patterns formed by using each of the semiconductor photoresist compositions according to Examples 1 to 7 exhibited superior sensitivity compared to those of Comparative Examples 1 to 4. For example, the semiconductor photoresist composition according to one or more embodiments, which included a compound including four or more hydroxyl groups and one or more ether bonds, was confirmed to exhibit excellent or suitable sensitivity.

In addition, the semiconductor photoresist compositions according to Examples 1 to 7 were each confirmed to exhibit no precipitates even after 5 days storage and no changes in viscosity and turbidity. In contrast, the photoresist composition of Comparative Example 1 was confirmed to exhibit precipitates after 5 days storage and thus changes in viscosity and turbidity.

It was confirmed that the semiconductor photoresist composition of one or more embodiments exhibited excellent or suitable storage stability.

In addition, the patterns formed by using each of the semiconductor photoresist compositions according to Examples 1 to 7 were confirmed to exhibit smaller surface roughness than those of Comparative Examples 1 to 4. For example, it was confirmed that the semiconductor photoresist composition according to one or more embodiments exhibited excellent or suitable coating characteristics.

In the present disclosure, expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof.

In the context of the present disclosure and unless otherwise defined, the terms “use,” “using,” and “used” may be considered synonymous with the terms “utilize,” “utilizing,” and “utilized,” respectively.

As utilized herein, the term “about,” or similar terms are used as terms of approximation and not as terms of degree, and are intended to account for the inherent deviations in measured or calculated values that would be recognized by those of ordinary skill in the art. “About” or “approximately,” as used herein, is also inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or ±5% of the stated value. Also, it should be understood that, even if the terms “about,” “approximately,” or “substantially” are not expressly recited in a given element (e.g., a claim element), the scope of such element is intended to include variations that are insubstantial or within the understanding of one of ordinary skill in the art. For example, numerical values and ranges provided herein are intended to include tolerances and measurement uncertainties that would be recognized by those skilled in the art, and the elements (e.g., claim elements) should be construed accordingly to encompass such equivalents.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

A person of ordinary skill in the art would appreciate, in view of the present disclosure in its entirety, that each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner unless otherwise stated or implied.

A pattern forming device, a semiconductor forming device and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random-access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

Hereinbefore, certain embodiments of the present disclosure have been described and illustrated, however, it is apparent to a person with ordinary skill in the art that the present disclosure is not limited to the embodiment as described, and may be variously modified and transformed without departing from the teachings and scope of the present invention. Accordingly, the modified or transformed embodiments as such may not be understood separately from the technical ideas and aspects of the present disclosure, and the modified embodiments are within the scope of the claims of the present disclosure. It is further to be understood that the scope of the present disclosure is defined by the appended claims and equivalents thereof rather than the detailed description described above, and all modifications and alterations derived from the claims and their equivalents fall within the scope of the present disclosure.

Reference Numerals

| | |
|---|---|
| 100: substrate | 102: thin film |
| 104: resist underlayer | 106: photoresist film |
| 106a: unexposed region | 106b: exposed region |
| 108: photoresist pattern | 112: organic film pattern |
| 110: patterned mask | 114: thin film pattern |

What is claimed is:

1. A semiconductor photoresist composition, comprising
an organometallic compound;
a compound comprising four or more hydroxyl groups and one or more ether bonds; and
a solvent.

2. The semiconductor photoresist composition as claimed in claim 1, wherein
the compound comprising four or more hydroxyl groups and one or more ether bonds comprises one or more compounds selected from among a saturated or unsaturated aliphatic compound and an aromatic compound, each comprising four or more hydroxyl groups and one or more ether bonds.

3. The semiconductor photoresist composition as claimed in claim 1, wherein
the compound comprising four or more hydroxyl groups and one or more ether bonds is a compound represented by Chemical Formula 1 or Chemical Formula 2: 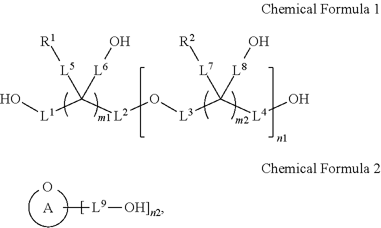
wherein, in Chemical Formula 1 and Chemical Formula 2,
L¹ to L⁹ are each independently a single bond or a substituted or unsubstituted C1 to C10 alkylene group,
R¹ and R² are each independently any one selected from among hydrogen, a hydroxyl group, a substituted or unsubstituted C1 to C10 alkyl group, a substituted or unsubstituted C2 to C10 alkenyl group, a substituted or unsubstituted C2 to C10 alkynyl group, a substituted or unsubstituted C1 to C10 heteroalkyl group, a substituted or unsubstituted C2 to C10 heteroalkenyl group, a substituted or unsubstituted C2 to C10 heteroalkynyl group, a substituted or unsubstituted C3 to C20 cycloalkane, a substituted or unsubstituted C3 to C20 cycloalkene, a substituted or unsubstituted C6 to C20 aromatic ring, and a substituted or unsubstituted C3 to C20 hetero aromatic ring,
A is any one selected from among a substituted or unsubstituted C4 to C20 oxacycloalkane and a substituted or unsubstituted C4 to C20 oxacycloalkene,
m1 and m2 are each independently one of the integers from 1 to 10,
n1 is one of the integers from 1 to 10, and
n2 is an integer greater than or equal to 4 and less than or equal to a valence of A.

4. The semiconductor photoresist composition as claimed in claim 3, wherein
in Chemical Formula 1, R¹ and R² are each independently any one selected from among hydrogen, a hydroxyl group, a substituted or unsubstituted C1 to C10 alkyl group, and a substituted or unsubstituted C3 to C20 cycloalkane.

5. The semiconductor photoresist composition as claimed in claim 3, wherein
in Chemical Formula 2, A is a substituted or unsubstituted C4 to C10 oxacycloalkane.

6. The semiconductor photoresist composition as claimed in claim 1, wherein
the compound comprising four or more hydroxyl groups and one or more ether bonds is a compound represented by any one selected from among Chemical Formula 1-1 to Chemical Formula 1-5:

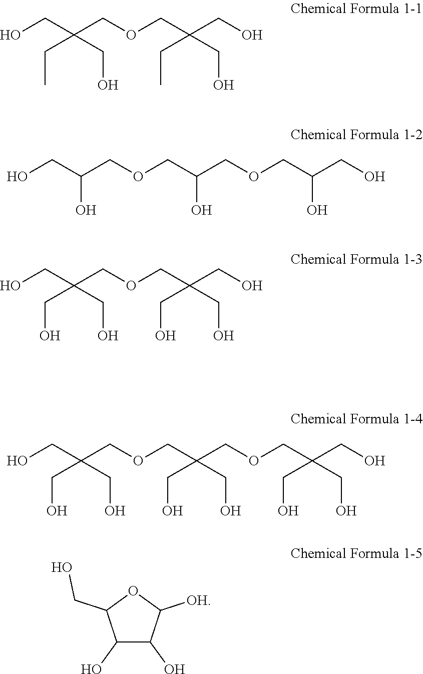 [SMILES: CCC(CO)(CO)COCC(CC)(CO)CO ; OCC(O)COCC(O)COCC(O)CO ; OCC(CO)(CO)COCC(CO)(CO)CO ; OCC(CO)(CO)COCC(CO)(CO)COCC(CO)(CO)CO ; OCC1OC(O)C(O)C1O]

7. The semiconductor photoresist composition as claimed in claim 1, wherein
the compound comprising four or more hydroxyl groups and one or more ether bonds is in an amount of 0.001 wt % to 10 wt % based on a total weight of 100 wt % of the semiconductor photoresist composition.

8. The semiconductor photoresist composition as claimed in claim 1, wherein
the organometallic compound is in an amount of 0.5 wt % to 30 wt % based on a total weight of 100 wt % of the semiconductor photoresist composition.

9. The semiconductor photoresist composition as claimed in claim 1, wherein
the semiconductor photoresist composition comprises an additive selected from among a surfactant, a crosslinking agent, a leveling agent, organic acid, quencher, and a combination thereof.

10. The semiconductor photoresist composition as claimed in claim 1, wherein
the organometallic compound is an organotin compound containing at least one of an organic oxy group or an organic carbonyloxy group.

11. The semiconductor photoresist composition as claimed in claim 1, wherein
the organometallic compound is represented by Chemical Formula 3: 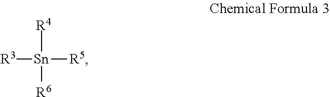
wherein, in Chemical Formula 3,
R³ is selected from among a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and a substituted or unsubstituted C7 to C30 arylalkyl group,
R⁴ to R⁶ are each independently a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, a substituted or unsubstituted C7 to C30 arylalkyl group, an alkoxy or aryloxy group (—OR^{a}, wherein R^{a} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl or acyloxy group (—O(CO)R^{b}, wherein R^{b} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylamido or dialkylamido group (—NR^{c}R^{d}, wherein R^{c} and R^{d} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidato group (—NR^{e}(COR^{f}), wherein R^{e} and R^{f} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidinato group (—NR^{g}C(NR^{h})Rⁱ, wherein R^{g}, R^{h}, and R′ are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylthio or arylthio group (—SR^{j}, wherein R^{j} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or a thiocarboxyl group (—S(CO)R^{k}, wherein R^{k} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and
at least one selected from among R⁴ to R⁶ is selected from among an alkoxy group and an aryloxy group (—OR^{a}, wherein R^{a} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and a carboxyl group and an acyloxy group (—O(CO)R^{b}, wherein R^{b} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

12. The semiconductor photoresist composition as claimed in claim 11, wherein
R⁴ to R⁶ are each independently selected from among an alkoxy group and an aryloxy group (—OR^{a}, wherein R^{a} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and a carboxyl group and an acyloxy group (—O(CO)R^{b}, wherein R^{b} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

13. The semiconductor photoresist composition as claimed in claim 12, wherein
R³ is a substituted or unsubstituted C1 to C8 alkyl group, a substituted or unsubstituted C3 to C8 cycloalkyl group, a substituted or unsubstituted C2 to C8 aliphatic unsaturated organic group comprising one or more double bonds or triple bonds, a substituted or unsubstituted C6 to C20 aryl group, a substituted or unsubstituted C4 to C20 heteroaryl group, a carbonyl group, an ethoxy group, a propoxy group, or a combination thereof,
R^{a} is a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof, and
R^{b} is hydrogen, a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof.

14. The semiconductor photoresist composition as claimed in claim 1, wherein
the organometallic compound is represented by Chemical Formula 4 or Chemical Formula 5: 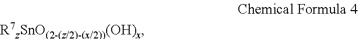
wherein, in Chemical Formula 4,
R⁷ is a C1 to C31 hydrocarbyl group, 0<z≤2, and 0<(z+x)≤4, and 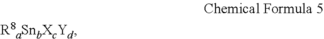
and
wherein, in Chemical Formula 5,
R⁸ is a substituted or unsubstituted C1 to C20 alkyl group, a substituted or unsubstituted C3 to C20 cycloalkyl group, a substituted or unsubstituted C2 to C20 aliphatic unsaturated organic group comprising one or more double bonds or triple bonds, a substituted or unsubstituted C6 to C30 aryl group, a substituted or unsubstituted C4 to C30 heteroaryl group, a carbonyl group, an ethylene oxide group, a propylene oxide group, or a combination thereof,
X is sulfur (S), selenium (Se), or tellurium (Te),
Y is —OR^{m} or —OC(═O)Rⁿ,
wherein R^{m} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof, and
Rⁿ is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof, and
a, b, c, and d are each independently an integer of 1 to 20.

15. A method, comprising
forming an etching-objective layer on a substrate;
applying the semiconductor photoresist composition as claimed in claim 1 to the etching-objective layer to form a photoresist film;
patterning the photoresist film to form a photoresist pattern; and
etching the etching-objective layer utilizing the photoresist pattern as an etching mask,
wherein the method is a method of forming patterns.

16. A system, comprising:
means for forming an etching-objective layer on a substrate;
means for coating the semiconductor photoresist composition of claim 1 on the etching-objective layer to form a photoresist film;
means for patterning the photoresist film to form a photoresist pattern; and
means for etching the etching-objective layer utilizing the photoresist pattern as an etching mask,
wherein the system is a system of forming patterns.

17. A system, comprising:
a coating module configured to form an etching-target layer on a substrate and to coat the semiconductor photoresist composition of claim 1 to form a photoresist film;
an exposure module configured to pattern the photoresist film;
a bake module configured to perform post-apply bake and post-exposure bake;
a developer module configured to develop the patterned photoresist film; and
an etching module configured to etch the etching-target layer utilizing the photoresist pattern as an etching mask,
wherein the system is a system of forming patterns.
